(19) United States
(12) Patent Application Publication
(10) Pub. No.: US 2011/0166226 A1
(43) Pub. Date: Jul. 7, 2011

(54) PROCESS FOR OBTAINING AN EXTRACT RICH IN ROSMARINIC ACID (RA) FROM THE PLANT ORIGANUM VULGARE AND ITS USE FOR THE TREATMENT OF DIABETES

(75) Inventors: Wison Roberto Cunha, Franca (BR); Rodrigo Lucarini, Ilicinea (BR); Maria Jose Silvino, Franca (BR); Marco Luis Andrade e Silva, Franca (BR); Ademar Alves Silva Filho, Franca (BR); Andréa Carla Celotto, Ribeirao Preto (BR); Carlos Henrique Gomes Martins, Ribeirao Preto (BR)

(73) Assignee: ACEF S/A, Franca - SP (BR)

(21) Appl. No.: 13/000,777

(22) PCT Filed: Jun. 25, 2009
(86) PCT No.: PCT/BR2009/000183

§ 371 Date: Mar. 21, 2011

(30) Foreign Application Priority Data
Jun. 25, 2008 (BR) PI0802688-2

(51) Int. Cl. A61K 31/225; C07C 69/76; A61P 3/10

(52) U.S. Cl. 514/548; 560/60

(57) ABSTRACT

Diabetes consists of a worldwide public health concern that leads to high levels of morbidity and premature mortality and affects millions of people in countries of any level of development. It is estimated that there are more than 150 million people with diabetes in the world, wherein projections from the World Health Organization (WHO) for 2025 such amount may get to 300 million cases.

The present invention refers to a process for obtaining an extract rich in rosmarinic acid (RA) from the plant Origanum vulgare and its use as a possible oral hypoglycemic agent for the treatment of diabetes.

The present invent refers to the process for obtaining rosmarinic acid (RA) and derivatives semi-synthetics from plant Origanum vulgare and their use for treatment of diabetes induced by alloxan, for a functional investigation of its bioactivity as an oral hypoglycemiant.

### STATE OF ART

Diabetes consists of a worldwide public health problem having high levels of morbidity and premature mortality, which affects millions of people in countries of any level of development.

It is estimated that there are more than 150 millions of people having diabetes in the world, wherein projections from World Health Organization (WHO) for 2025 such amount may get to 300 millions.

In the United States of America, the disease reaches about 16 millions of Americans, around 2 to 4% of population, having more than 700,000 new cases being diagnosed every year. There, diabetes is the third cause of mortality and morbidity, having an average of 40,000 deaths and 20,000 amputations a year. It is estimated that more than 100 billions of dollars are annually spent by American government for treating diabetes and its complications.

Brazil presents a prevalence of diabetic subjects similar to more developed countries, having the aggravating that 46% of sick people are not early diagnosed and 22% have no treatment of any kind.

Diabetes is a hereditary metabolic syndrome of multiple etiologies, characterized by a state of persistent hyperglycemia, which results from a deficiency in the production of insulin or from the resistance of the tissues to the action of such hormone. There has been shown that environmental factors stimulate the gene expression in a variable way, thus justifying the different ages for the symptoms appearance of the disease. The interaction of such factors seems to be also involved in reinforcing this disease's pathological expression.

Insulin is a polypeptidic hormone produced by cells β from Islands of Langerhans in pancreas. Its main function is to control the intermediate metabolism, by operating on the liver, muscle, and adipose tissue. The global effect of insulin consists of maintaining the energetic fuel when facilitating glucose, lipid, and protein attraction, use, and storage. Although there are several complications diabetic subjects present, little is known on such disease etiology, and it is due to several factors such as: age when the disease appears, and the incidence in the several ethnic groups. However, several attempts have been done in order to elucidate how, when, why, and which deleterious effects in the organic systems and if they may be reverted with treatment or diets.

The main groups of hypoglycemiant agents that reduce glycemia are sulfonylurea, biguanides, and the inhibitors like α-glycosidase. The sulfonylurea has the main action on the β cells, stimulating insulin secretion and, thus, reducing the plasma concentrations of glucose. However, in order to have such function, they demand the presence of functional β cells. It is indicated for thin patients, who have insulin deficiency in higher level. Its side effects are hypoglycemia and body weight increase.

The main drugs found in the market are: tolbutamide, glybenclamide, chlorpropamide. Biguanide group need no functional β cells. Its function is complex and has not been fully clarified. It is known they increase the sensitivity to insulin, reduce intestinal absorption of glucose, reduce gluconeogenesis and increase glucose attraction by peripheral tissues. The main non-desirable effect consists of transitory gastrointestinal disturbances. The intestinal α-glucosidase inhibitors operate by retarding carbohydrate absorption, reducing postprandial glycemia increasing. The most common side effects are flatulence, soft feces, diarrhea, abdominal pain, and distension. Both biguanide and intestinal α-glucosidase inhibitors are indicated for patients having type 2 diabetes who are obese and do not respond the treatment only through diet.

Several pathogenic processes are involved in the development of diabetes and its complications. As consequence of hyperglycemia and disturbances, such condition causes in metabolism of carbohydrates, protein and lipid, most of patients having diabetes manifest in short-time clinical form of glucosuria, ketosis and ketonuria, polyphagia, polydipsia, and polyuria. These symptoms, which are frequent in diabetic people, are known as classical in such disease history. However, the absence of them is common in many patients with diabetes and do not leave the possibility that there are a level of hyperglycemia enough to cause functional or pathological changes before the diagnosis is done.

Nevertheless, long-term consequences of diabetes include dysfunction and several organs collapse, specially kidney, eyes, nerves, heart, and blood vessels. Among those, the development of cardiovascular diseases has been considered the main cause for life reduction and for mortality of diabetic patients.

Diabetes conventional therapy done with repeated applications of insulin, diet and frequent monitoring of glucose level in blood and in urine, extensively restores metabolic control, however, alternative methods by employing natural origin products have been studied.

Perhaps, the search for relief and cure of diseases by herbs and leaves ingestion has been one of the first ways of using natural products. The history of development of eastern and western civilizations is rich in examples of the use of natural resources in medicine. The deep knowledge of the chemical arsenal from nature, by primitive peoples and by natives may be considered a fundamental factor to discover toxic and medicinal substances throughout time. The bioprospection of new active principles from superior plants to be used as prototypes in the development of new drugs has been a constant practice of researchers last decades.

Origanun vulgare (oregano) is a plant known by its medicinal value, being officially accepted in a number of countries. Its flowers and leaves are extensively used in homeopathy. Its essential oil is used in Indian traditional medicine as stimulator and fortifier aroma. However, it has a limited use in perfume production and cosmetics.

Although the ancients put together different species under the same name, oregano is considered an aromatic plant essential for medical and culinary use since old times. Theophrastus, Aristotle, and Hippocrates praised its benefic action in breathing diseases, ulcers, burnings and weak digestion.

An ethnopharmacological literature attributes to such plant properties of nervous system, string analgesic action, spasmolytics, sudorific, digestion stimulator, uterine activity, as well as light expectorant.

Oregano's essential oil has a wide spectrum in vivo and in vitro, as potential antimicrobial, antifungal, insecticidal, antioxidant, and with anticarcinogenic activity. The phenolic monoterpens are the highest constituent responsible for such biological actions. Studies evidenced the antioxidant activity and anti-inflammatory action of the extract prepared with oregano. In general, the substances biologically active extracted from plants are the secondary metabolites, which have important role in the mechanism of chemical defense, and the one emphasized in the present invention is the rosmarinic acid, one of the major components present in Origanum vulgare.

Rosmarinic acid is a secondary metabolite, connected to a group esters and heteroside substances phenolic acids and cinnamic acid. Such substances present wide distribution in vegetal reign, being found as esters, glycoside and amide. In this group the derivative of cafeic acid. Such secondary metabolite is commonly found in Lamiaceae and Boraginaceae family.

Rosmarinic acid is an ester from cafeic acid and lactic acid (3,4 dihydroxiphenil), being isolated for the first time from the specie Rosmarinus officinalis by two Italian chemists. Several biological activities have been described for rosmarinic acid, being the main: antimicrobial, antiviral and antioxidant. They present actions against rheumatism, anti-inflammatory action, anticarcinogenic action, anti-allergy actions, antioxidants, anti-inflammatory, antipoison (antidote), antidepressant and suppressor. In recent studies, rosmarinic acid is emphasized as anti-HIV action property. Nevertheless, in scientific literature there is no report that relates the hypoglycemiant activity of rosmarinic acid, such fact that motivated investigation on its potential as a hypoglycemiant.

Many species of plants have been used in a etnopharmacologycal way or in experimental way in order to treat the symptoms of diabetes (Oliveira, 1989; Ivorra et al., 1989; Rahman, Zaman, 1989; Handa, Chawla, 1989; Neef et al., 1995; Johns, Chapman, 1995; Marles, Farnsworth, 1995; Ernst, 1997; Pereira, 1997; Kar et al., 1999, 2003; Lamba et al., 2000; Novaes et al., 2001; Mccune, Jonhns, 2002; Said et al., 2002; Volpato et al., 2002; Grover et al., 2002b; Syem et al., 2002; Huo et al., 2003; Elder, 2004; Saxena, Vikram, 2004). Such plants represent more than 725 genders in 183 families, physiologically extending from seaweed and fungus to plants. The phylogenetics distance between such group of families is a strong indication of the varied nature of its active constituents. Most of plants used as anti-diabetics, when pharmacologically evaluated, present hypoglycemiant activity and chemical constituents that may be used as models for new hypoglycemiant agents. However, later analysis showed great variety of action mechanisms that may take to the hypoglycemiant effect.

The action mechanism from which plants reduce blood glucose tax may be attributed to the following factors: increase in releasing insulin through stimulation of β-pancreatic cells; resistance to hormones that increase glucose tax; increase of number and sensitivity of the insulin receptor site; decrease of loss of de glycogen; increase of consumption of glucose in tissues and organs; elimination of free radicals; resistance to lipid peroxidation; correction of the metabolic disorder caused in lipids and proteins and stimulus to blood microorganism increase in the organism. Although several drugs are used to control diabetes, the perfect glycemic control is rarely reached. Thus, new alternatives of safer and more efficient therapeutics are highly important in order to overcome existing problems.

In this context, this investigation aims at evaluating the activity of infusion hypoglycemiant, hydroalcoholic extract from Origanum vulgare, as well as isolated substance—rosmarinic acid (RA) in diabetic rats, induced by alloxan and the investigation of its bioactivity. At the end of the experience, important dada were obtained, which may provide subside to perform diabetes conventional and alternative therapy, including the development of an oral hypoglycemiant.

### SHORT DESCRIPTION OF THE INVENTION

The invention characterizes by the process for obtaining and the use of infusion, raw hydroalcoholic extract obtained from plant Origanum vulgare, isolated substance—rosmarinic acid (RA) and its derivatives semi-synthetics.

The raw extract, infusion and, mainly, the isolated substance, the rosmarinic acid (RA), have the property of reducing the level of plasmatic glycemic in diabetic rats and not reducing the glycemic level in normal rats. According to the used experimental model, the results indicate an application of the use of the substance RA and/or extracts of O. vulgare in type 1 and 2 diabetes treatment.

Today, due to the great number of diabetic people, the search for efficient alternatives having few side effects and low cost is a still distant reality. Nevertheless, vegetal reign has been showing a huge potential as alternative for treatment of such pathology, mainly type 2 one.

### DESCRIPTION OF DRAWINGS

The present invention is better understood with the attached figures provided as examples but not limited to, in which:

FIG. 1—General flowchart of the procedure for obtaining raw hydroalcoholic extracts from O. vulgare;

FIG. 2—General flowchart of the procedure for extracting acid from O. vulgare;

FIG. 3—General flowchart of the procedure for experimental inducing of diabetes by alloxan; treatment of experimental model;

FIG. 4—Graphic of the variation in plasmatic levels of glycemia (mg/dL), observed in the different experimental groups: Diabetic Control (DC), Diabetic treated with hydroalcoholic extract (DTE, 250 mg/Kg, v.o.) and Normoglycemic Control (NC);

FIG. 5—Graphic of the variation in plasmatic levels of glycemia (mg/dL), observed in the different experimental groups: Diabetic Control (DC), Diabetic Treated with Rosmarinic Acid (DTRA, 25 mg/kg, v.o.) and Normoglycemic Control (NC);

FIG. 6—Graphic of the variation in plasmatic levels of glycemia (mg/dL), observed in the different experimental groups: Diabetic Control (DC), Diabetic Treated with Infusion (DTC, 55 mL/rat, v.o.) and Normoglycemic Control (NC);

FIG. 7—Graphic of the variation in plasmatic levels of glycemia (mg/dL), observed in the different experimental groups: Diabetic Control (DC), Diabetic treated with Infusion (DTC, 55 mL/rat, v.o.), Diabetic treated with hydroalcoholic extract (DTE, 250 mg/kg, v.o.) and Diabetic treated with Rosmarinic Acid (DTRA, 25 mg/kg, v.o.), and

FIG. 8—Graphic of the variation in plasmatic levels of glycemia (mg/dL), observed in the different experimental groups: Diabetic treated with hydroalcoholic extract (DTE, 250 mg/kg, v.o.), Diabetic treated with Rosmarinic Acid (DTRA, 25 mg/kg, v.o.), Diabetic treated with drugs in the market (Clorpropamida, 40 mg/kg), Diabetic Control (DC) and Normoglycemic Control (NC).

### DETAILED DESCRIPTION OF THE INVENTION

As it can be observed through figures, the extract has been prepared from leaves and branches from O. vulgare. The vegetal was dried and stabilized in a greenhouse with warm circulating air at about 40° C. Afterwards, it was grinded to powder knife mill. The vegetal resulting powder went through exhaustive extraction by maceration with ethanol/water (95:5 v/v) at room temperature. It was performed three successive extractions, having a week interval between them. All material resulting from the process of maceration was filtered and concentrated under pressure reduced to 60° C. by means of a rotary evaporator until the solvent full elimination. Dry vegetal extract was stored in amber bottle with a lid and maintained in refrigerator until the moment of experiments execution.

For preparing the infusion, it was used 20 g of leaves from O. vulgare for 1 liter of water at 100° C. The boiling water was poured on the leaves; the container was covered, being kept like this for 30 minutes, so that the active substances from the leaves could have been extracted. After this time, infusion was filtered in a paper filter, and, then, provided to the animals, being prepared every days of the treatment.

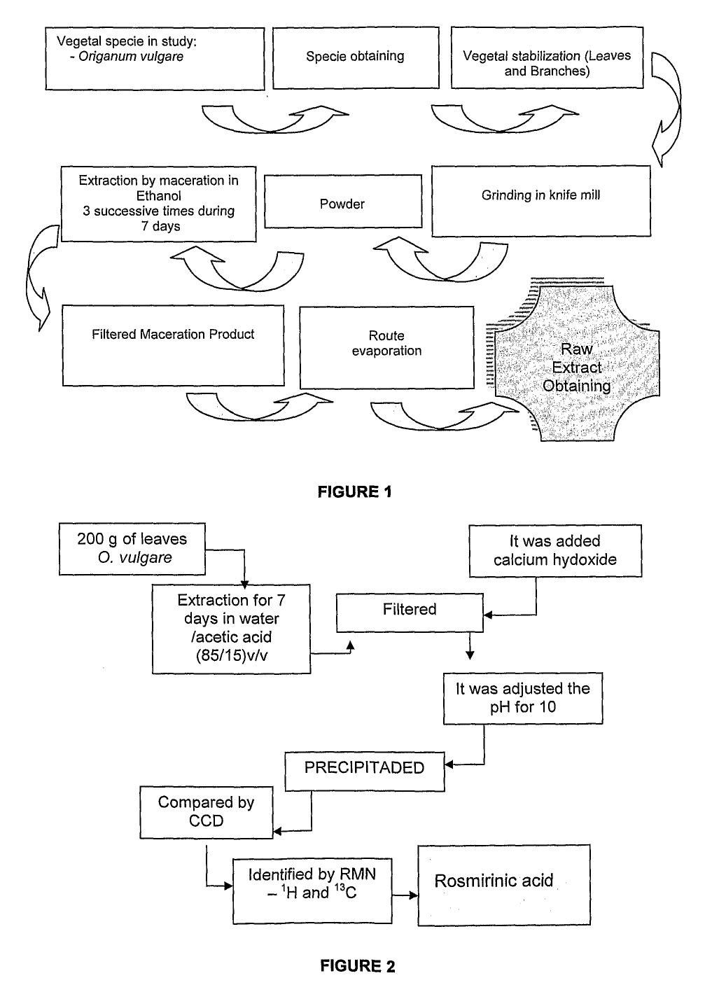

For isolating the rosmarinic acid (RA) it was used 200 g of powder of leaves from the vegetal O. vulgare. It was submitted to the process of extraction by maceration (room temperature) during seven days using water/acetic acid (Merck) (85:15 v/v). The maceration product was filtered and the pH adjusted to 10, by adding a solution of calcium hydroxide. It has been formed, then, a precipitated that was identified by comparing the authentic pattern to be the RA (FIG. 2/Tanaka et al, 2001). The final identification was performed by Hydrogen and Carbon Nuclear Magnetic Resonance. (RMN—¹H and ¹³C) of the composition.

Although several experimental models of diabetes promotion are available, the most frequently used is the chemical diabetes induction by delivering toxic agents like Alloxan in rodents.

Diabetogenic activity of Alloxan was initially observed by DUNN et al. (1943), when he studied the effects of uric acid and its derivatives in the production of renal lesion in rabbits. Alloxan is a cytotoxic beta pancreatic agent, and has contributed for most of information related to human diabetes. The diabetogenic drug provokes three-phase answer in glycemic levels during the first hours of its delivering and, in 24 hours, it establishes permanent diabetes.

Thus, the option was this chemical method of endocrine suppression of pancreas, which exhibits all biochemical, hormonal, and morphological events that occur during and after induction of the diabetogenic state. Alloxan is a chemical agent having cytotoxicity specific for beta cells, most studied.

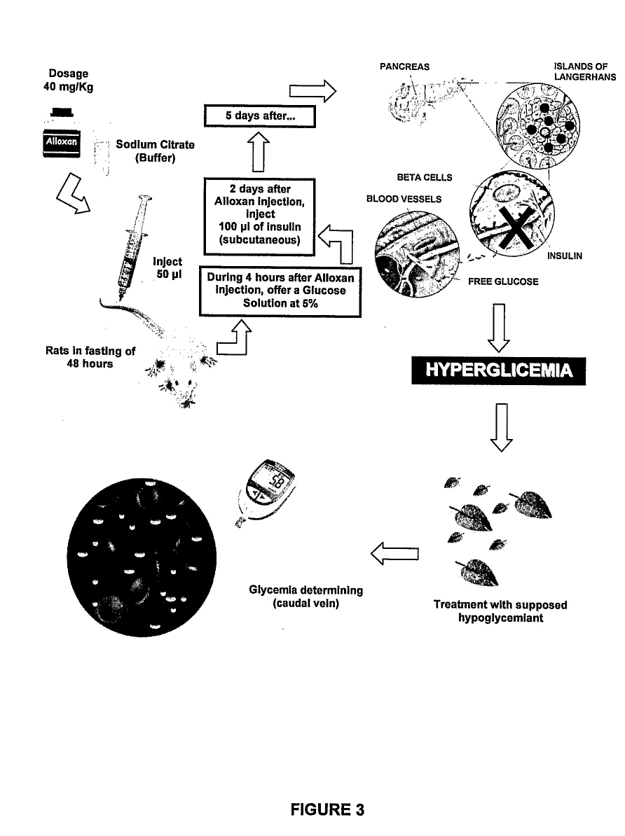

The induction of diabetes in animals was done by using alloxan. The animals stayed in fasting for 24 hours before receiving injections of alloxan, so that the animals became more susceptive to diabetes. After a pilot study, the dose of alloxan that was used was 40 mg/kg injected via intravenous in caudal vein. Alloxan was diluted in sodium citrate 0.05M pH 4.5 and the injected volume was 500. During the four hours after the injection, the animals received glucose solution 5% via oral (ad libidum) in order to prevent from seizures and death, what is common in hypoglycemia. On the first two days after diabetes induction, the animals received an insulin injection (100 μl—diluted 1/10) at every 24 hs via subcutaneous (FIG. 3). This procedure guarantees animals to survive during the disease acute phase enabling the study. On the fifth day after alloxan injection, animals' glycemia was evaluated and just the animals that presented glycemia superior or equals to 250 mg/dl.

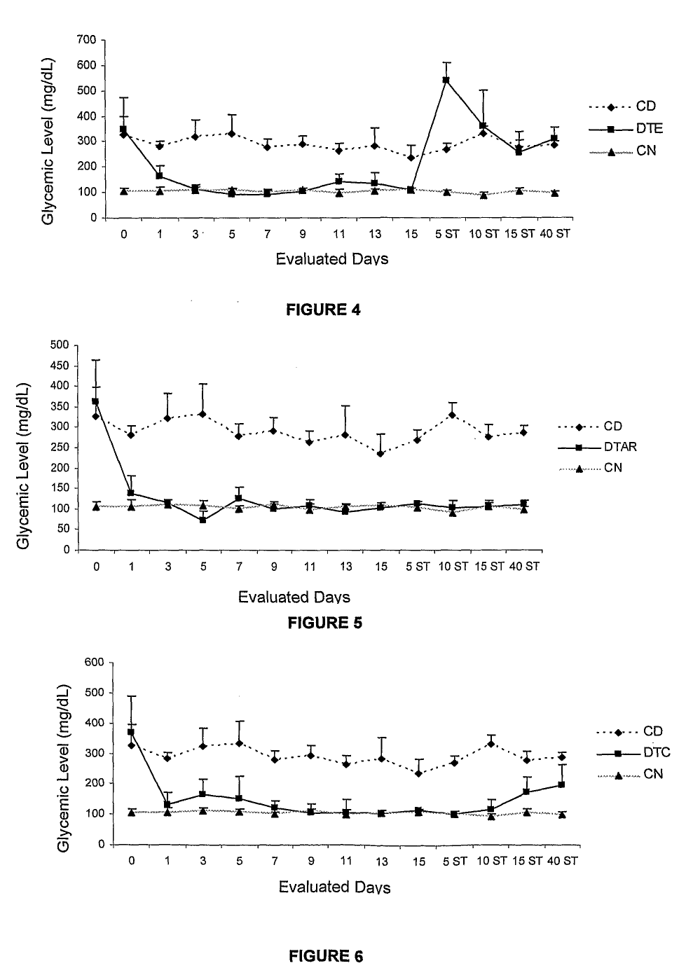

To use in the animals, it was employed corresponding doses, considering animals body mass, which is about 250 mg/kg of hydroalcoholic extract, 55 mL of infusion/rat and 25 mg/kg of RA. The animals received treatment via oral (v.o.), being maintained during a period of 40 days. The first 15 days evaluated by receiving treatment and the subsequent days with no treatment, so that it was possible to evaluate the potential to maintain the glycemic level. The obtained results are represented in the charts from FIGS. 4 to 7.

As it can be observed in FIG. 4, the glycemic levels were monitored during 15 alternate days. After 15 days, the treatment was suspended (ST) and it was evaluated during 5, 10, 15 and 40 days. The data represent the mean±EPM, P<0.0001, when compared to the answer obtained for the groups DTE versus CD and CD versus CN (Anova followed by the Turkey-Kramer test).

In FIG. 5, glycemic levels were monitored during 15 alternate days. After 15 days, the treatment was suspended (ST) and it was evaluated during 5, 10, 15 and 40 days. The data represent the mean±EPM, P<0.0001, when compared to the answer obtained for the groups DTAR versus CD and CD versus CN (Anova followed by the Turkey-Kramer test).

In FIG. 6, glycemic levels were monitored during 15 alternate days. After 15 days, the treatment was suspended (ST) and it was evaluated during 5, 10, 15 and 40 days. The data represent the mean±EPM, P<0.0001, when compared to the answer obtained for the groups DTC versus CD and CD versus CN (Anova followed by the Turkey-Kramer test).

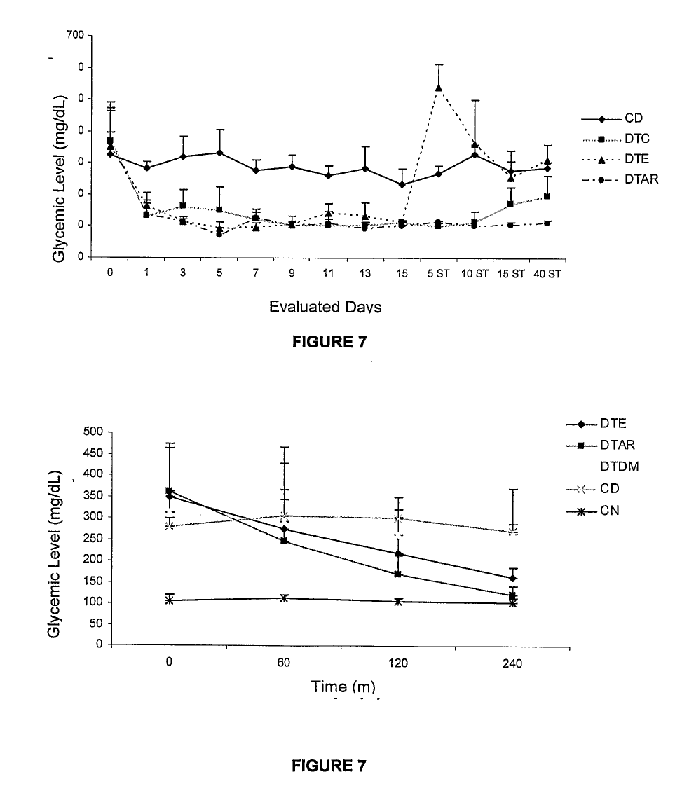

In FIG. 7, glycemic levels were monitored during 15 alternate days. After 15 days, the treatment was suspended (ST) and it was evaluated during 5, 10, 15 and 40 days. The data represent the mean±EPM, P<0.0001, when compared to the answer obtained for the groups treated versus CD. (Anova followed by the Turkey-Kramer test).

In FIG. 8, glycemic levels were monitored after 60, 120 and 240 minutes. The data represent the mean±EPM, P<0.0001, when compared to the answer obtained for the groups treated (DTE, DTAR, DTDM) and control (CD and CN)-Anova followed by the Turkey-Kramer test.

Rosmarinic acid (Scheme 1—chemical structure 1) was the active composition that represented the best results (chart from FIG. 5), in addition of reducing, it kept the glycemic level to the end of the experience. However, it is believed its semi-synthetic derivatives (Scheme 1-chemical structures 2, 3, and 4) present also potentially active as hypoglycemiants. In the experience where it was monitored the glycemia for 4 h, by using chlorpropamide as positive control, it was verified the effectiveness of the isolated substance rosmarinic acid (chart from FIG. 8). In table 1 there are all the results in the charts from FIGS. 4-7.

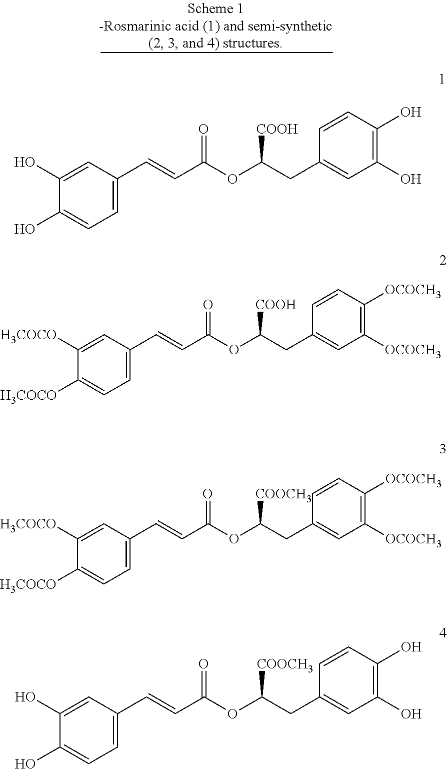

The results obtained may be observed in the table hereinbelow:

TABLE 1
Results of hypoglycemiant activity in diabetic rats induced by alloxan, treated
with infusion, extract from vegetal specie O. vulgare and isolated substance - AR

| | Treated | Treated | | | |
|---|---|---|---|---|---|
| Evaluated | Diabetic | Diabetic | Treated | | Normoglycemic |
| Days | Infusion | Extract | Diabetic RA* | Diabetic Control | Control |
| 0 | 368.5 ± 122.23 | 350.0 ± 123.89 | 362.6 ± 101.52 | 326.8 ± 70.08 | 104.6 ± 13.01 |
| 1 | 130.6 ± 41.63 | 162.6 ± 42.47 | 136.6 ± 44.96 | 281.1 ± 22.01 | 105.1 ± 16.73 |
| 3 | 162.6 ± 50.82 | 113.0 ± 16.41 | 114.0 ± 8.46 | 320.5 ± 63.28 | 110.8 ± 11.08 |
| 5 | 151.1 ± 72.46 | 91.3 ± 23.29 | 71.5 ± 22.66 | 331.0 ± 74.91 | 107.0 ± 11.76 |
| 7 | 122.0 ± 23.36 | 94.3 ± 17.62 | 124.5 ± 27.75 | 277.3 ± 31.34 | 99.5 ± 7.94 |
| 9 | 105.1 ± 28.09 | 103.1 ± 14.16 | 100.1 ± 12.31 | 290.1 ± 34.00 | 110.0 ± 8.39 |
| 11 | 103.8 ± 42.48 | 141.6 ± 30.21 | 108.0 ± 13.31 | 262.5 ± 29.56 | 97.8 ± 14.90 |
| 13 | 104.8 ± 8.93 | 133.1 ± 41.76 | 92.1 ± 16.89 | 281.3 ± 70.98 | 105.0 ± 8.46 |
| 15 | 112.5 ± 5.54 | 109.8 ± 9.30 | 102.0 ± 12.60 | 234.6 ± 48.37 | 107.1 ± 8.93 |
| 5 ST** | 102.0 ± 7.79 | 541.5 ± 71.48 | 112.5 ± 5.00 | 268.0 ± 36.70 | 101.0 ± 7.42 |
| 10 ST | 114.6 ± 32.67 | 361.5 ± 140.30 | 101.5 ± 19.13 | 330.0 ± 50.30 | 89.5 ± 8.42 |
| 15 ST | 172.6 ± 50.30 | 256.3 ± 83.58 | 103.5 ± 10.78 | 275.3 ± 11.12 | 103.6 ± 10.70 |
| 40 ST | 196.0 ± 64.19 | 311.3 ± 46.86 | 109.3 ± 9.87 | 285.1 ± 23.20 | 98.2 ± 13.12 |

*AR - rosmarinic acid;
**ST - no treatment

There is not in the market drugs, whether phytotherapic or not, that are efficient in reducing the glycemic level and maintain it. Thus, the products of the present invention will provide the development of an innovative oral hypoglycemiant.

1. A process for obtaining an extract rich in rosmarinic acid and semi synthetics derivatives from the plant Origanum vulgare, comprising:
a) drying aerial parts, leaves, and branches of Origanum in a greenhouse with warm circulating air at 40° C. to produce dried material,
b) grinding the dried material in knife mills to produce powder,
c) extracting at least part of the powder with water/acetic acid solvent mixture (85:15 v/v) for 7 days,
d) lyophylization of aqueous extract, treating the water/acetic acid solution (85:15 v/v) with calcium hydroxide for separation of the extract rich in rosmarinic acid (RA) as a solid.

2-7. (canceled)

8. A method of treating diabetes comprising administering the extract obtained by the process of claim 1 to a patient in need thereof.

9. An extract obtained by the process of claim 1.

10. The extract of claim 9, wherein the extract is an oral hypoglycemic agent.
